(19) United States
(12) Patent Application Publication
(10) Pub. No.: US 2013/0317542 A1
(43) Pub. Date: Nov. 28, 2013

(54) STEERABLE DELIVERY SYSTEM

(71) Applicant: BOSTON SCIENTIFIC SCIMED, INC., Maple Grove, MN (US)

(72) Inventors: Christopher J. Clark, St. Michael, MN (US); David J. Holtan, Eden Prairie, MN (US)

(21) Appl. No.: 13/900,590

(22) Filed: May 23, 2013

Related U.S. Application Data
(60) Provisional application No. 61/651,576, filed on May 25, 2012.

(51) Int. Cl. A61B 17/00

(52) U.S. Cl. CPC A61B 17/0057
USPC 606/213

(57) ABSTRACT

The disclosure pertains to a delivery system for delivering a left atrial appendage treatment device or the like to a left atrium and methods of use therefor. The delivery system includes a control housing containing a differential displacement mechanism adapted to manipulate the distal end of an elongate steerable catheter through differential displacement of control elements attached proximate the distal end of the elongate steerable catheter. The elongate steerable catheter includes a stiff proximal region, a somewhat more flexible curved intermediate region and a more flexible distal region adapted to be displaced from the plane of the second curved region.

### BACKGROUND

The present disclosure pertains to transseptal access systems for accessing the left atrium from the right atrium, such as by crossing the fossa ovalis, for the purpose of delivering a left atrial appendage treatment device.

The typical human heart includes a right ventricle, a right atrium, left ventricle, and left atrium. The right atrium is in fluid communication with the superior vena cava and the inferior vena cava. On the inner wall of the right atrium, where it is separated from the left atrium, is a thin walled, recessed portion, the fossa ovalis. In the heart of a fetus, the fossa ovalis is open (patent foramen) permitting fetal blood to flow between the right and left atria, bypassing the fetal lungs in favor of the placental blood flow. In most individuals, this opening closes after birth. In as many as about 20 percent of adults an opening (the patent foramen) still remains in place of the fossa ovalis between the right and left atria.

A wide variety of diagnostic and therapeutic procedures have been developed in which a catheter is transluminally advanced into various chambers and across valves of the heart. The most difficult chamber of the heart to access with a catheter is the left atrium. Access to the left atrium through the pulmonary artery is not possible. Approaches from the left ventricle are difficult, may cause arrhythmias and may present difficulty in obtaining stable catheter positioning. Accordingly, the presently preferred method of accessing the left atrium is through a transseptal approach, achieved by catheterization of the right atrium with subsequent penetration of the interatrial septum. The reduced wall thickness and location of the fossa ovalis makes it a useful access point for a transseptal access.

The objectives of left atrial access can be either diagnostic or therapeutic. Presently, the therapeutic objectives of left atrial access are primarily three-fold. The first is mitral valvuloplasty which represents an alternative to surgical procedures to relieve obstruction of the mitral valve. The second therapeutic objective is for electrophysiological intervention in the left atrium. More recently, therapeutic treatment of the left atrial appendage (LAA) to reduce the risk of embolic stroke has also been proposed. Such treatments may block or filter blood flow between the left atrium and the LAA.

In patients with atrial fibrillation, blood tends to pool and form clots in an area of the heart called the left atrial appendage. The LAA is a pouch-like extension located in the upper left chamber of the heart. A blood clot that breaks loose from this area may migrate through the blood vessels and eventually plug a smaller vessel in the brain or heart resulting in a stroke or heart attack. Clinical studies show that the majority of blood clots in patients with atrial fibrillation originate in the LAA.

Despite clinical acceptance of a wide variety of procedures which require access to the left atrium, significant room for improvement remains in the actual access technique. Once access to the left atrium has been achieved, it is still necessary to direct interventional devices to an appropriate portion of the left atrium, for example into the left atrial appendage, the position and structure of which is somewhat variable.

### SUMMARY

This disclosure pertains to a delivery system for a left atrial appendage treatment device, the delivery system comprising an elongate steerable catheter having a proximal end, a distal end, and a plurality of lumens extending at least partially therebetween, wherein at least one lumen of the elongate steerable catheter is sized and adapted to slidably receive a left atrial appendage treatment device; a first control member and a second control member each having a proximal end, a distal end, and being adapted to occupy a first control member lumen and a second control member lumen of the elongate steerable catheter, said first and second control members being affixed at their respective distal ends to attachment points proximate the distal end of the elongate steerable catheter and circumferentially displaced from each other by approximately 180 degrees, wherein the elongate steerable catheter comprises a first generally straight section, a second section including a first radius of curvature and defining a reference plane; and a third section having a first configuration in which it is approximately coplanar with the second section and a second configuration in which the third section includes a portion which is curved, having a second radius of curvature less than the first radius of curvature, and lies in a plane which is generally perpendicular to the reference plane; and a control housing attached to the proximal end of the elongate steerable catheter and having a lumen therethrough which is generally coaxial with the at least one lumen sized and adapted to slidably receive a left atrial appendage treatment device of the elongate steerable catheter; and a differential displacement mechanism associated with the control housing, wherein the first and second control members are adapted to be differentially displaced by the differential displacement mechanism, further wherein differential displacement of the first and second control members by the differential displacement mechanism is adapted to move the third section between the first configuration and the second configuration.

This disclosure also pertains to a method of delivering a left atrial appendage treatment device to a left atrial appendage comprising inserting through a vascular puncture a delivery system comprising an elongate steerable catheter having a plurality of lumens extending at least partially therethrough, first and second control members which occupy first and second lumens of the elongate steerable catheter and which are attached to a distal end of the elongate steerable catheter at points approximately 180 degrees apart, and a control housing having a differential displacement mechanism adapted to impart differential motion to the first and second control members thereby moving a third section between a first configuration and a second configuration; advancing the elongate steerable catheter through the vena cava to the right atrium; positioning the distal end of the elongate steerable catheter adjacent to the fossa ovalis; crossing the fossa ovalis; manipulating the differential displacement mechanism to the position the distal end of the elongate steerable catheter proximate the ostium of a left atrial appendage; confirming the position of the distal end of the elongate steerable catheter relative to the ostium of the left atrial appendage; advancing a left atrial appendage treatment device through a lumen of the elongate steerable catheter sized and adapted to slidably receive a left atrial appendage treatment device to a position distal of the distal end of the elongate steerable catheter; treating the left atrial appendage; removing the elongate steerable catheter from the vasculature; and sealing the vascular puncture.

### BRIEF DESCRIPTION OF DRAWINGS

FIG. 1 illustrates an embodiment of the disclosure.

FIG. 2 illustrates a partial cutaway view of the embodiment of FIG. 1 rotated 90 degrees.

FIG. 3 is an axial cross-sectional view of the catheter portion of an embodiment of the disclosure schematically depicted in straightened form.

FIG. 4 is a transverse cross-sectional view of the catheter of FIG. 4.

FIG. 5 is a perspective view of a detail of an embodiment of the disclosure.

FIGS. 6A-6D are schematic views of an embodiment of the disclosure as it is positioned in a left atrial appendage.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments whether or not explicitly described unless clearly stated to the contrary.

As used herein, the term “radius of curvature” is to be interpreted broadly so as to include a mean radius of curvature for a section of a catheter which departs from a linear configuration. That is, the specification of a section having a radius of curvature should not be read as requiring that the section necessarily is limited to a circular arc. Such sections may describe portions of other curves. Similarly, “coplanar” and “perpendicular” are intended to be broadly indicative of relative orientation and thus as including departures from the strict meaning of the terms. It will be further understood that each of these terms describes features whose form and orientation may vary somewhat during use as the features interact with the body and are manipulated during use.

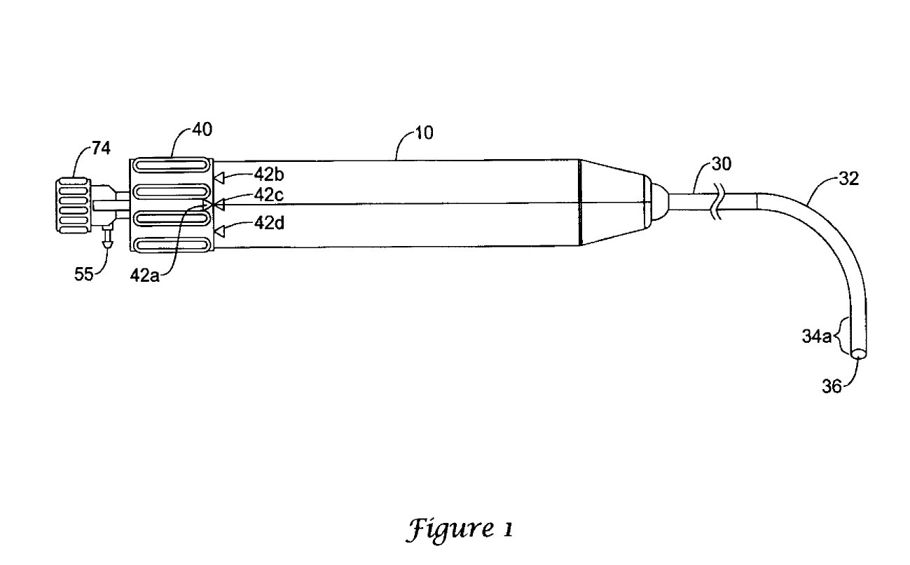

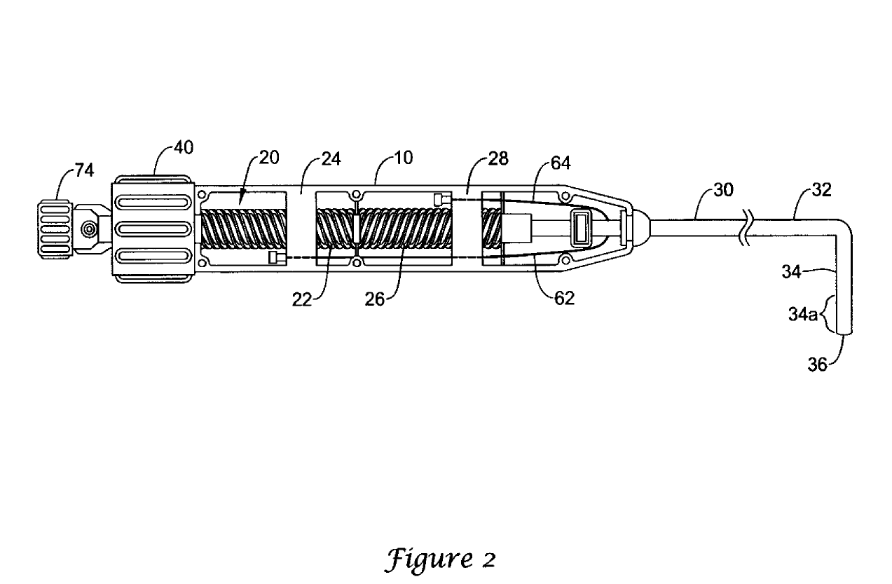

FIGS. 1 and 2 show views of an embodiment of the disclosure in which a first orientation of a delivery system is presented in FIG. 1 and a partial cutaway view of the delivery system which has been rotated by 90 degrees is presented in FIG. 2. The delivery system includes a control housing 10, an elongate steerable catheter having three sections 30, 32, and 34 attached thereto, and a differential displacement mechanism 20 within control housing 10 which is adapted to move the third section 34 of the elongate steerable catheter between a first configuration and a second configuration. As depicted in FIGS. 1 and 2, the third section 34 is in the second configuration. Rotation of knob 40 relative to control housing 10 rotates joined left-hand threaded segment 22 and right-hand threaded segment 26 of a tubular element of differential displacement mechanism 20 relative to housing 10 thereby imparting differential linear motion to left-hand follower 24 and right-hand follower 28 relative to housing 10. This in turn imparts differential displacement to the connected proximal ends of control members 62 and 64 which engage their respective followers. It will be appreciated that the illustrated differential displacement mechanism 20 is illustrative of various mechanisms which are known in the art for imparting differential displacement and that alternate differential displacement mechanisms are also contemplated. In contrast to single displacement control mechanisms which depend upon the resilience of components of the system to return a catheter to the initial configuration when tension is relaxed, a differential displacement mechanism is capable of providing positive displacement and positive return to a desired final configuration.

The illustrated differential displacement mechanism also is capable of providing significant mechanical advantage for the deflection of distal end 36, fine control of the catheter deflection, and may include a delivery system lumen 74 therethrough which extends continuously from a proximal end of the control housing and through the elongated steerable catheter to facilitate the use of a guidewire (not shown) to advance the elongate steerable catheter through the vasculature and/or to facilitate delivery of a piercing implement (not shown), left atrial appendage treatment device (not shown), or other medical device through the delivery system. Lumen 74 is sized and adapted to slidably receive the left atrial appendage treatment device or other medical device with which the delivery system is to be used. Lumen 74 may be in fluid communication with proximal fluid access port 55. Lumen 74 may include one or more hemostatic seals and/or additional ports as is common in the art.

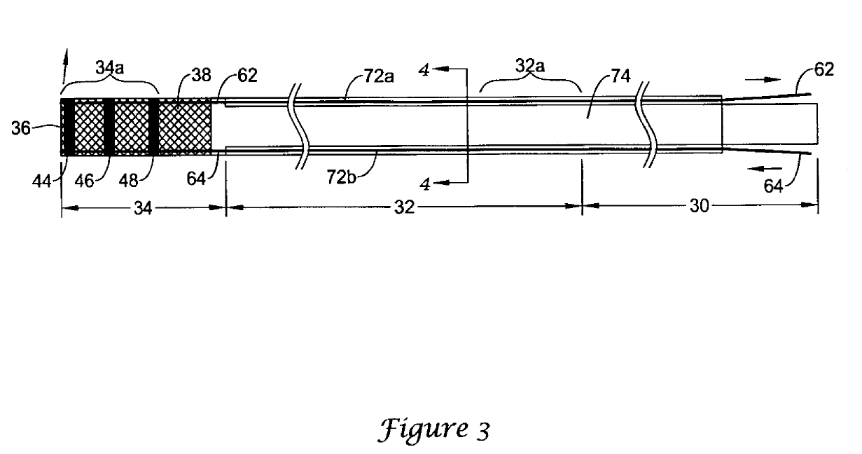

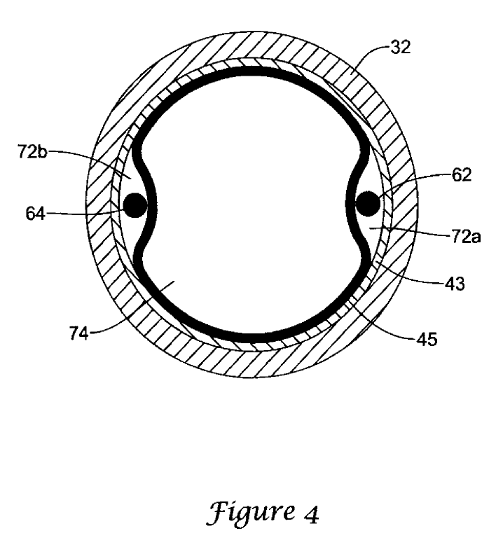

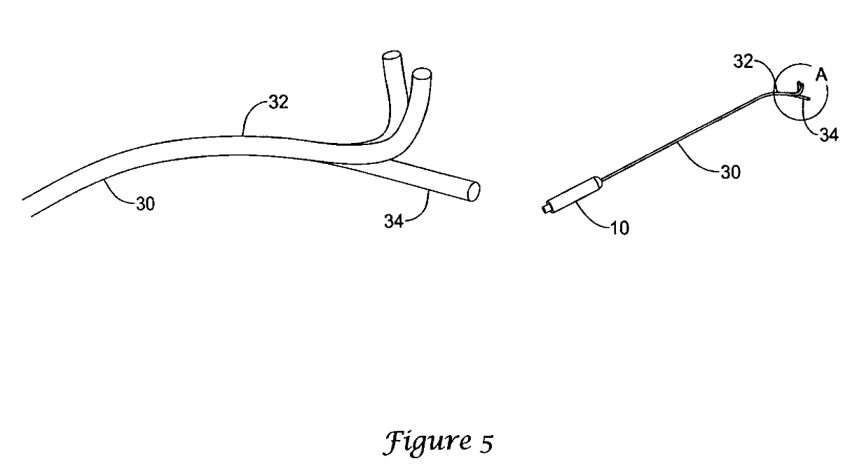

Control members 62 and 64 pass through first and second lumens 72a, 72b (see FIGS. 3 and 4) of the elongate steerable catheter and as shown their distal ends are attached to the distal end 36 of third section 34 of the elongate steerable catheter. First and second lumens 72a, 72b terminate distally near the transition between sections 32 and 34 of the elongate steerable catheter such that the differential displacement imparted to control members 62 and 64 by differential displacement mechanism 20 is imparted in turn to the distal end 36 of third section 34 of the elongate steerable catheter causing it to deflect relative to second section 32 as illustrated in FIG. 5 which is a detail of the delivery system as indicated in the inset. Lumens 72a and 72b may be formed by any of the conventional means known in the art such as by extrusion, molding, by bonding tubular members such as polyimide tubing to the interior of the elongate tubular catheter, or, as illustrated, by the providing one or more interior layers 43, 45 within elongate steerable catheter sections 30 and 32 with gaps therebetween to form lumens 72a and 72b. In some embodiments, one or both of interior layers 43 and 45 may be formed of a lubricious liner material to facilitate motion of control members 62 and 64 as well as the passage of the left atrial appendage treatment device or other medical device with which the delivery system is to be used through lumen 74. It will be appreciated that in certain embodiments it may be desirable to include additional lumens for conventional purposes such as aspiration, infusion of contrast medium, and the like.

Knob 40 and control housing 10 may include indicia 42a-42d, illustrated somewhat schematically in FIG. 1 as alignable arrows, which serve to indicate the degree of displacement which has been imparted to the distal end 36 of third section 34 of the elongate steerable catheter by turning the knob 40. Other forms of indicia are contemplated and may include indications of multiple turns of knob 40.

As illustrated in FIG. 3, in which the various sections of the elongate steerable catheter are not necessarily to scale, the elongate steerable catheter includes a first generally straight section 30 which forms the bulk of the catheter body and which is attached at its proximal end to the control housing 10, a second intermediate section 32 which typically includes a curved region having a first radius of curvature and which optionally may include a straight portion 32a, and an actuatable third section 34 which also may include an optional straight section 34a. Distal end 36 of third section 34 may be provided with an atraumatic tip if desired. In addition, third section 34 may optionally include a coil or braid 38 which minimizes the tendency of third section 34 to collapse as the section is bent by differential motion of control members 62, 64 which are attached to the distal end 36 of the third section 34 at points separated circumferentially by about 180 degrees. The points of attachment for control members 62, 64 to the distal end 36 of the third section 34 lie in a plane which is generally perpendicular to a reference plane defined by the curved portion of intermediate section 32 such that differential motion of control members 62, 64 cause third section 34 to curve in a plane generally perpendicular to the reference plane defined by the curved portion of intermediate section 32.

It should be understood that in the first configuration of the third section 34, the end 36 will typically lie in or near the reference plane defined by the curved portion of intermediate section 32 and will approximate a linear extension of the distal end of intermediate section 32, while in the second configuration, end 36 will be more removed from the reference plane as the third section 34 assumes a curved shape having a second radius of curvature which is less than the radius of the curved portion of second intermediate section 32. The second configuration of third section 34 represents a capability of the delivery system which may not be fully utilized in the course of any particular instance of a use of the delivery system as the degree of deflection which is necessary to align the distal end 36 of the third section 34 with a left atrial appendage will depend upon the anatomy of the left atrium.

When the approach path to the right atrium is through the inferior vena cava, the various curves associated with the elongate steerable catheter may be described as follows. If the first proximal section 30 is positioned along the X-axis of a right-hand Cartesian coordinate system such that the distal end lies in the +X direction, the distal end of the second intermediate section 32 will lie in the X, Y plane and extend in the −Y direction. When the delivery system has been actuated to place the third section 34 in the second configuration, the distal end 36 will extend from the X, Y plane in the +Z direction.

In some embodiments, the differential displacement mechanism may be adapted to move the third section 34 to a third configuration which is substantially the mirror image of the second configuration such that the distal end 36 of third section 34 will extend from the X, Y plane in the −Z direction. In other embodiments, the differential displacement mechanism may be adapted to move the distal end 36 of third section 34 to either the second or third configuration as well as any intermediate positions. In such embodiments, it may be especially desirable to include multiple indicia, such as 42a-42d, which serve to indicate both the direction of deflection as well as the degree of displacement which has been imparted to the distal end 36 of third section 34 of the elongate steerable catheter by turning knob 40.

Structurally, elongate steerable catheter typically will be formed to include a proximal region having a first flexural modulus, a proximal end, and a distal end, an intermediate region having a second flexural modulus, a proximal end, and a distal end and wherein the distal region has a third flexural modulus and a proximal end such that the distal end of the proximal region is directly joined to the proximal end of the intermediate region and the distal end of the intermediate region is directly joined to the proximal end of the distal region and the first flexural modulus is greater than or equal to the second flexural modulus and the second flexural modulus is greater than the third flexural modulus. If desired, the first, second and third regions may be joined by gradual transitions having intermediate flexural moduli. The flexural moduli of the regions may be imparted by selection of materials and/or structural features such as wall thickness or reinforcing elements. In some embodiments, the first proximal region will generally correspond to the first generally straight proximal section 30 and will be designed to have good pushability and torqueability. It will be appreciated that in certain embodiments, the elongate steerable catheter may include additional regions.

The second intermediate region will generally correspond to the second section 32 including a first radius of curvature. The curvature may be introduced by means known by those of skill in the art and may include, for example, forming or molding the section around a mandrel or bending and heat setting an initially straight section. This section typically will have a degree of flexibility and resilience which allows the catheter to track a guidewire or guide catheter through the vasculature and subsequently to return to the original curvature within the right atrium to position the distal end 36 of third section 34 adjacent the fossa ovalis.

The distal region will generally correspond to the third section 34 and as discussed previously will typically flex readily under the influence of differential displacement of control elements 62, 64, but will resist kinking either through material selection or as the result of the inclusion of a coil or braid 38 within the distal region. Optional radiopaque markers 44, 46, 48 may be used to judge the degree to which the third section 34 has entered the left atrial appendage.

It will be appreciated that although the proximal, intermediate and distal regions generally correspond to the respective first, second and third sections 30, 32, 34 as described and illustrated herein, in some embodiments it may be desirable to somewhat displace the boundaries between regions to improve the overall mechanical performance of the elongate steerable catheter. For example, the somewhat stiffer proximal region may be allowed to extend at least partially into the curved second section 32 thereby improving the ability of that part of the curve to position the third section. It will be further appreciated that in certain embodiments, the elongate steerable catheter may include additional regions.

In use, the delivery system is typically introduced into the vasculature in the conventional manner, as for example through the right femoral vein. Access may also be achieved through a puncture in any of a variety of other veins of suitable interior diameter, for example the left femoral vein, and the present disclosure is not limited in this regard.

In some embodiments, a conventional spring tipped guidewire is advanced through an introducer sheath along the right femoral vein and through the inferior vena cava to the right atrium. Prior to insertion of the delivery system, lumen 74 will typically be flushed with saline or other liquid through proximal fluid access port 55 of control handle 10 to remove air from the device. Subsequently, the delivery system of the disclosure is advanced over the guidewire to the right atrium whereupon the guidewire may be removed and replaced with a transseptal piercing implement (not shown) which is directed to the fossa ovalis by the curvature of the second section 32 of the elongated steerable catheter. In the alternative, a guide catheter may be initially positioned within the right atrium and the delivery system may be advanced therewithin to a position adjacent to the fossa ovalis.

Once the fossa ovalis has been pierced by the piercing implement, the distal end 36 of the elongate steerable catheter may cross the fossa ovalis to be inserted transseptally into the left atrium. Removal of the piercing implement before or after further advancing the delivery device into the left atrium clears the lumen 74 of the delivery system to allow a left atrial appendage treatment device, diagnostic devices, or other medical devices to be inserted and advanced to a position proximate the distal end 36 of the delivery system. It will be appreciated that other techniques may be used to deliver the transseptal delivery system to the left atrium or to other locations in the body.

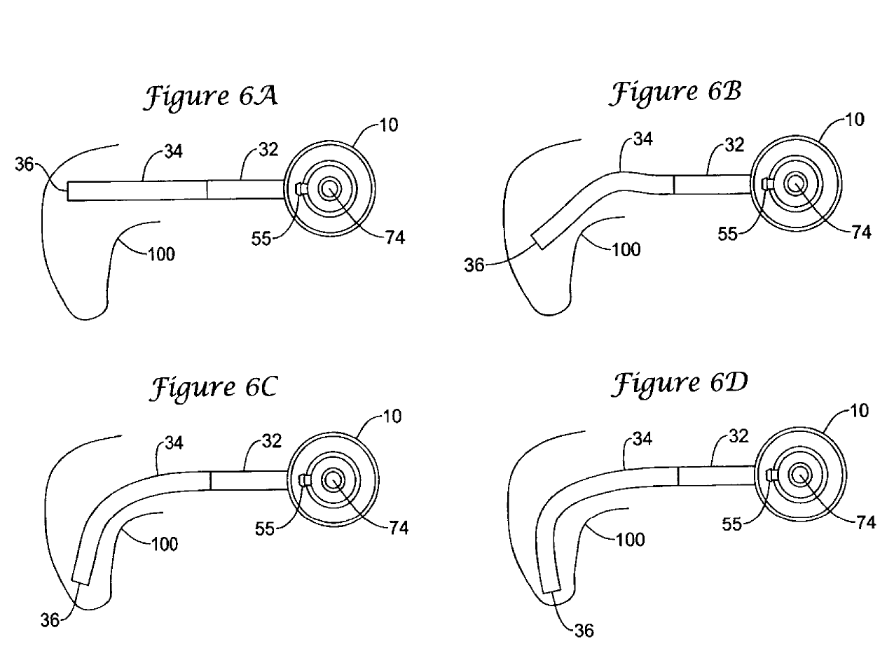

The distal end 36 of the third section 34 of the elongate steerable catheter may then be advanced and directed to the ostium of the left atrial appendage, or other target if desired, by advancing the control housing 10, torqueing the control housing 10, and/or rotating knob 40 relative to the control housing 40. As noted previously, indicia 42a-42b, or the like, may be used to judge the degree of deflection of third section 34 relative to the reference plane defined by the curved portion of second section 32. Distal end 36 of the third section 34 of the elongate steerable catheter may then be advanced into the left atrial appendage 100 as shown in the sequence illustrated in FIGS. 6A-6D by further manipulation of the delivery system. Optional radiopaque markers 44, 46, 48 may be used to judge the degree to which the third section has entered the left atrial appendage 100.

FIGS. 6A-6D are highly schematic in that features of the anatomy other than the left atrial appendage 100 have been removed for clarity. In each of the views, the proximal end of control handle 10 and lumen 74 are shown such that first generally straight section 30 is hidden by control handle 10 which extends away from the viewer into the plane of the paper and through the vasculature to the inferior vena cava where second section 32 curves through the right atrium and the fossa ovalis (not shown) and across the left atrium.

Once distal end 36 is positioned within the left atrial appendage 100, contrast media may be injected through proximal fluid access port 55, or other port, to confirm proper positioning within the left atrial appendage 100. A left atrial appendage treatment device, diagnostic devices, or other medical devices may be inserted and advanced to a position proximate the distal end 36 of the delivery system within the left atrial appendage 100 whereupon the distal end 36 of third section 34 may be withdrawn relative to the left atrial appendage treatment device, diagnostic devices, or other medical devices to effect treatment of the left atrial appendage 100. The elongate steerable catheter may then be withdrawn from the vasculature and the vascular puncture may be sealed.

Although the illustrative examples described above relate to a delivery system for a left atrial appendage treatment device, delivery of other medical devices is also contemplated as are other target portions of the anatomy. In such embodiments, at least a portion of one lumen of the elongate steerable catheter is sized and adapted to slidably receive the medical device to be delivered.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. A delivery system for a left atrial appendage treatment device, the delivery system comprising:
an elongate steerable catheter having a proximal end, a distal end, and a plurality of lumens extending at least partially therebetween,
wherein at least one lumen of the elongate steerable catheter is sized and adapted to slidably receive a left atrial appendage treatment device;
a first control member and a second control member each having a proximal end, a distal end, and being adapted to occupy a first control member lumen and a second control member lumen of the elongate steerable catheter, said first and second control members being affixed at their respective distal ends to attachment points proximate the distal end of the elongate steerable catheter and circumferentially displaced from each other by approximately 180 degrees,
a control housing attached to the proximal end of the elongate steerable catheter and having a lumen therethrough which is generally coaxial with the at least one lumen sized and adapted to slidably receive a left atrial appendage treatment device of the elongate steerable catheter; and
a differential displacement mechanism associated with the control housing,
wherein the first and second control members are adapted to be differentially displaced by the differential displacement mechanism.

2. The delivery system of claim 1, wherein the elongate steerable catheter comprises a first generally straight section, a second section including a first radius of curvature and defining a reference plane; and a third section having a first configuration in which it is approximately coplanar with the second section and a second configuration in which the third section includes a portion which is curved, having a second radius of curvature less than the first radius of curvature, and lies in a plane which is generally perpendicular to the reference plane; and
further wherein differential displacement of the first and second control members by the differential displacement mechanism is adapted to move the third section between the first configuration and the second configuration.

3. The delivery system of claim 2 wherein the elongate steerable catheter includes a proximal region having a first flexural modulus, a proximal end, and a distal end, an intermediate region having a second flexural modulus, a proximal end, and a distal end, and a distal region having a third flexural modulus and a proximal end such that the distal end of the proximal region is joined to the proximal end of the intermediate region and the distal end of the intermediate region is joined to the proximal end of the distal region and the first flexural modulus is greater than or equal to the second flexural modulus and the second flexural modulus is greater than the third flexural modulus.

4. The delivery system of claim 2, wherein displacement mechanism comprises a tubular element having a right-hand threaded segment, a left-hand threaded segment, and having an axial lumen therethrough which is coaxial with the at least one lumen of the elongate steerable catheter sized and adapted to slidably receive a left atrial appendage treatment device and the control housing.

5. The delivery system of claim 4, wherein the displacement mechanism further comprises a right-hand threaded follower adapted to engage the right-hand threaded segment and a left-hand threaded follower adapted to engage the left-hand threaded segment.

6. The delivery system of claim 5, wherein the first control member is operably connected to the right-hand threaded follower and the second control member is operably connected to the left-hand threaded follower.

7. The delivery system of claim 2, wherein the first lumen and the second lumen of the elongate steerable catheter terminate at their respective distal ends between the second section and the third section of the elongate steerable catheter.

8. The delivery system of claim 2, further comprising a differential displacement indicator adapted to indicate a degree of differential displacement attained by the differential displacement mechanism.

9. The delivery system of claim 2, wherein the third section has a third configuration in which the third section includes a second radius of curvature less than the first radius of curvature and lies in a plane which is generally perpendicular to the reference plane, said third configuration positioning the third section on a side opposite the reference plane from the second configuration of the third section,
wherein differential displacement of the first control member and second control member by the differential displacement mechanism is adapted to move the third section between the first configuration and the third configuration.

10. The delivery system of claim 2, wherein the third section includes one of a coil or a braid adapted to prevent collapse of the at least one lumen of the third section when the third section is in one of the second or third configurations.

11. The delivery system of claim 2, wherein the third section includes one or more radiopaque markers.

12. The delivery system of claim 2, wherein the proximal end of the elongate steerable catheter is in fluid communication with a fluid access port.

13. The delivery system of claim 2, wherein the at least one lumen of the elongate steerable catheter includes a lubricious liner.

14. The delivery system of claim 2, wherein the second section of the elongate steerable catheter further includes a linear portion.

15. The delivery system of claim 2, wherein the third section of the elongate steerable catheter further includes a linear portion.

16. The delivery system of claim 3, wherein the proximal region generally corresponds to the first generally straight section.

17. The delivery system of claim 3, wherein the intermediate region generally corresponds to the second section.

18. The delivery system of claim 3, wherein the distal region generally corresponds to the third section.

19. A method of delivering a left atrial appendage treatment device to a left atrial appendage comprising:
inserting through a vascular puncture a delivery system comprising an elongate steerable catheter having a plurality of lumens extending at least partially therethrough, first and second control members which occupy first and second lumens of the elongate steerable catheter and which are attached to a distal end of the elongate steerable catheter at points approximately 180 degrees apart, and a control housing having a differential displacement mechanism adapted to impart differential motion to the first and second control members thereby moving a third section between a first configuration and a second configuration;
advancing the elongate steerable catheter through the vena cava to the right atrium;
positioning the distal end of the elongate steerable catheter adjacent to the fossa ovalis;
crossing the fossa ovalis;
manipulating the differential displacement mechanism to the position the distal end of the elongate steerable catheter proximate the ostium of a left atrial appendage;
confirming the position of the distal end of the elongate steerable catheter relative to the ostium of the left atrial appendage;
advancing a left atrial appendage treatment device through a lumen of the elongate steerable catheter sized and adapted to slidably receive a left atrial appendage treatment device to a position distal of the distal end of the elongate steerable catheter;
treating the left atrial appendage;
removing the elongate steerable catheter from the vascular puncture; and
sealing the vascular puncture.

20. The method of claim 19, wherein the elongate steerable catheter, the control housing, and the differential displacement mechanism of the delivery system define the lumen adapted to slidably receive the left atrial appendage treatment device, said lumen extending from a proximal end of the control housing to the distal end of the elongate steerable catheter.
